(19) United States
(12) Patent Application Publication
(10) Pub. No.: US 2023/0143524 A1
(43) Pub. Date: May 11, 2023

(54) A NOVEL STRAIN OF LACTOBACILLUS REUTERI LM1071 SEPARATED FROM BREAST MILK,AND COMPOSITION FOR RELIEVING PREMENSTRUAL SYNDROME COMPRISING THE STRAIN OR ITS CULTURE FLUID

(71) Applicant: LACTOMASON CO., LTD., Jinju-si, Gyeongsangnam-do (KR)

(72) Inventors: Minn SOHN, Jinju-si, Gyeongsangnam-do (KR); Tae Rahk KIM, Suwon-si, Gyeonggi-do (KR); Woo Jung PARK, Gangneung-si, Gangwon-do (KR)

(21) Appl. No.: 17/796,315

(22) PCT Filed: Jan. 21, 2022
(86) PCT No.: PCT/KR2022/001091

§ 371 Date: Jul. 29, 2022

(30) Foreign Application Priority Data
Feb. 9, 2021 (KR) 10-2021-0018249

(51) Int. Cl. A61K 35/747; A61P 29/00

(52) U.S. Cl. CPC A61K 35/747; A61P 29/00

(57) ABSTRACT

The present disclosure relates to a strain of Lactobacillus reuteri LM1071 (KCCM12650P) and a composition for relieving menstrual pain, comprising the same.

A strain of Lactobacillus reuteri LM1071 (KCCM12650P) according to an embodiment of the present disclosure can relieve premenstrual syndrome by reducing NO, prostaglandin E2 and leukotriene in blood and increasing epoxyeicosatrienoic acid (EET) and the production ratio of prostaglandin E1/prostaglandin E2. Therefore, the strain can be applied to food compositions, health functional food compositions, pharmaceutical compositions and the like.

### TECHNICAL FIELD

The present disclosure relates to a strain of Lactobacillus reuteri LM1071 (KCCM12650P) separated from breast milk and a composition for relieving premenstrual syndrome comprising the strain.

### BACKGROUND

Premenstrual syndrome (PMS) refers to a group of symptoms characterized by emotional, behavioral and physical symptoms that occur repeatedly before menstruation and includes various physical symptoms such as menstrual pain and psychological changes such as mood swings, feeling depressed, anxious or aggressive. These symptoms become progressively worse after ovulation and are most severe one week before menstruation and disappear within a few days of the onset of menstruation.

Among the typical physical symptoms, menstrual pain is a common gynecological symptom that every woman of childbearing age experiences periodically every month until she reaches menopause and is a very common symptom in the female menstrual cycle.

According to a study, 77% to 94% of Korean women complained of menstrual pain. Also, 47% of these women experienced menstrual pain every month and 53.2% experienced severe menstrual pain. Further, 46% of female manufacturing workers reported that they were restricted from their activities due to menstrual pain. In foreign countries, the prevalence of menstrual pain was as high as 60% to 93%, and 42% of the respondents experienced severe menstrual pain. Furthermore, 10% to 50% of female students reported that menstrual they were restricted from their daily activities including school activities due to menstrual pain. In the industrial fields, damage caused by menstrual pain was estimated at 600 million hours of work loss and $2 billion of productivity loss every year.

Therefore, the results of studies on the changes or mechanisms of menstrual pain-related factors can be used as sufficient evidence for the development of premenstrual syndrome-related food materials, health functional food materials and drugs.

A composition for improving premenstrual syndrome symptom comprising compounds isolated from Hordeum vulgare extract (Korean Patent No. 10-2187335) is an example of a composition developed for relieving premenstrual syndrome through the change of prolactin. However, there is still a need for in-depth development and various studies on compositions having excellent effects of relieving premenstrual syndrome.

Accordingly, the present inventors made diligent efforts to develop an excellent composition capable of relieving premenstrual syndrome and resultantly developed a novel strain capable of reducing NO, prostaglandin E2 and leukotriene and increasing epoxyeicosatrienoic acid (EET) and the production ratio of prostaglandin E1/prostaglandin E2 in blood, and completed the present disclosure.

### DISCLOSURE OF THE INVENTION

### Problems to be Solved by the Invention

The present disclosure provides a novel strain of Lactobacillus reuteri LM1071 (KCCM12650P) separated from breast milk and a composition for relieving premenstrual syndrome, comprising the strain.

However, problems to be solved by the present disclosure are not limited to the above-described problems. Although not described herein, other problems to be solved by the present disclosure can be clearly understood by a person with ordinary skill in the art from the following description.

### Means for Solving the Problems

A first aspect of the present disclosure provides a strain of Lactobacillus reuteri LM1071 (KCCM12650P).

A second aspect of the present disclosure provides a food composition for relieving menstrual pain, comprising one or more of a strain of Lactobacillus reuteri LM1071 (KCCM12650P), and culture fluid, fragments and extracts of the strain as active ingredients.

A third aspect of the present disclosure provides a pharmaceutical composition for preventing or treating premenstrual syndrome, comprising one or more of a strain of Lactobacillus reuteri LM1071 (KCCM12650P), and culture fluid, fragments and extracts of the strain as active ingredients.

### Effects of the Invention

A strain of Lactobacillus reuteri LM1071 (KCCM12650P) according to an embodiment of the present disclosure can relieve premenstrual syndrome by reducing NO in blood, prostaglandin E2 and leukotriene and increasing epoxyeicosatrienoic acid (EET) and the production ratio of prostaglandin E1/prostaglandin E2. Therefore, the strain can be applied to food compositions, health functional food compositions, pharmaceutical compositions and the like.

### BRIEF DESCRIPTION OF THE DRAWINGS

FIG. 1 shows changes in amount of nitric oxide (NO) secreted from macrophage cells by Lactobacillus reuteri LM1071.

FIG. 2 shows changes in amount of PGE2 secreted from macrophage cells by Lactobacillus reuteri LM1071.

FIG. 3 shows changes in amount of PGE1 secreted from macrophage cells by Lactobacillus reuteri LM1071.

FIG. 4 shows changes in ratio of PGE1/PGE2 from macrophage cells by Lactobacillus reuteri LM1071.

FIG. 5 shows changes in secretion amount of LTB4 by Lactobacillus reuteri LM1071.

FIG. 6 shows changes in secretion amount of EET by Lactobacillus reuteri LM1071.

### BEST MODE FOR CARRYING OUT THE INVENTION

Hereinafter, embodiments of the present disclosure will be described in detail with reference to the accompanying drawings so that the present disclosure may be readily implemented by a person with ordinary skill in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “on” that is used to designate a position of one element with respect to another element includes both a case that the one element is adjacent to the other element and a case that any other element exists between these two elements.

Through the whole document, the term “comprises or includes” and/or “comprising or including” used in the document means that one or more other components, steps, operation and/or existence or addition of elements are not excluded in addition to the described components, steps, operation and/or elements unless context dictates otherwise. Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

Hereinafter, embodiments and examples of the present disclosure will be described in detail with reference to the accompanying drawings. However, the present disclosure may not be limited to the following embodiments, examples, and drawings.

A first aspect of the present disclosure provides a strain of Lactobacillus reuteri LM1071 (KCCM12650P).

In an embodiment of the present disclosure, the strain may reduce the production amount of NO in blood.

Through the whole document, the term “NO (nitric oxide)” refers to a compound in which nitrogen is oxidized, which is called “nitrogen oxide” or “nitrogen monoxide”. It is formed from arginine, which is an amino acid, in cells, and serves as a kind of signal transmitter and is involved in various physiological activities such as immune reaction, vasodilation and signal transmission. It is also known to induce inflammation and pain by promoting the secretion of inflammatory cytokines such as TNF-α and IL-6 (Hu et al., 2020).

Accordingly, it can be seen that the strain of Lactobacillus reuteri LM1071 of the present disclosure can reduce the production amount of NO and thus relieve menstrual pain.

In an embodiment of the present disclosure, the strain may reduce prostaglandin E2 in blood.

Through the whole document, the term “prostaglandin” refers to a kind of hormone secreted during menstruation and serves to contract the uterus and causes menstrual blood to be smoothly pushed out when the endometrium is peeled off.

Prostaglandin is converted from arachidonic acid by a cyclooxygenase (COX). Prostaglandin possessing various physiological activities is produced through conversion from arachidonic acid into prostaglandin G2 and then into prostaglandin H2. Also, prostaglandin has been reported to cause inflammation and pain (Jayesh et al., 2020).

Through the whole document, the term “PGE2 (prostaglandin E2)”, also known as “dinoprostone”, is a naturally occurring prostaglandin with oxytocic properties.

PGE2 acts through G protein-coupled receptors that induce inflammation-mediated pain in damaged tissues or cells (Treutlein et al., 2018) and stimulate the central nervous system and the peripheral nervous system (Grφsch et al., 2017).

Accordingly, it can be seen that the strain of Lactobacillus reuteri LM1071 of the present disclosure can reduce prostaglandin E2 in blood and thus relieve menstrual pain.

In an embodiment of the present disclosure, the strain may increase the ratio of prostaglandin E1/prostaglandin E2 in blood. Specifically, it can be seen that the strain increases the production ratio of prostaglandin E1 to prostaglandin E2.

Through the whole document, the term “PGE1 (prostaglandin E1)”, also known as “alprostadil”, is a naturally occurring prostaglandin which serves to dilate blood vessels in the body, and is known to induce smooth muscle relaxation and attenuate the secretion of inflammatory cytokines and thus relieve pain (Gezginci-Oktayoglu et al., 2016).

The production ratio of PGE1/PGE2 (PGE1 to PGE2) is a major indicator according to the guidelines from the Ministry of Food and Drug Safety for women with premenstrual syndrome.

An increase in the production ratio is considered to have a function related to treatment of premenstrual syndrome.

Accordingly, it can be seen that the strain of Lactobacillus reuteri LM1071 of the present disclosure can increase the production ratio of prostaglandin E1/prostaglandin E2 in blood and thus relieve menstrual pain and premenstrual syndrome.

In an embodiment of the present disclosure, the strain may reduce leukotriene in blood.

Through the whole document, the term “leukotriene (LT)” refers to one of a family of eicosanoid inflammatory mediators produced through arachidonic acid metabolism by 5-lipoxygenase and is known to induce inflammatory responses and vascular damage (Tunctan et al., 2019). Accordingly, studies for reliving inflammation and pain through inhibition of arachidonic acid metabolism have been continuously reported (Cheng et al., 2019; Jayesh et al., 2020).

Accordingly, it can be seen that the strain of Lactobacillus reuteri LM1071 of the present disclosure can reduce leukotriene in blood and thus relieve menstrual pain.

In an embodiment of the present disclosure, the strain may increase epoxyeicosatrienoic acid (EET) in blood.

Through the whole document, the term “epoxyeicosatrienoic acid (EET)” refers to a signaling molecule formed through two pathways in an unsaturated fatty acid having 20 carbons, such as arachidonic acid. EET is known to suppress an increase of inflammatory cytokines such as IL-6 by inhibiting the activity of NK-kB and mediate inflammation by promoting the activity of peroxisome proliferator-activated receptor (PPAR) (Tunctan et al., 2019).

Accordingly, it can be seen that the strain of Lactobacillus reuteri LM1071 of the present disclosure can increase epoxyeicosatrienoic acid (EET) in blood and thus relieve menstrual pain.

In an embodiment of the present disclosure, the strain may relieve menstrual pain. Specifically, the strain may be contained in various compositions such as a food composition, a health functional food composition and a pharmaceutical composition for relieving menstrual pain.

A second aspect of the present disclosure provides a food composition for relieving menstrual pain, comprising one or more of a strain of Lactobacillus reuteri LM1071 (KCCM12650P), and culture fluid, fragments and extracts of the strain as active ingredients. The features described above in respect of the first aspect of the present disclosure may equally apply to the food composition according to the second aspect of the present disclosure.

Through the whole document, the term “relieve” refers to all activities reducing menstrual pain or improving related symptoms by administering the composition.

In an embodiment of the present disclosure, the composition may relieve menstrual pain. Specifically, the composition may relieve menstrual pain by reducing NO, prostaglandin E2 and leukotriene in blood and increasing epoxyeicosatrienoic acid (EET) and the production ratio of prostaglandin E1/prostaglandin E2 in blood.

In an embodiment of the present disclosure, the composition may contain a strain of Lactobacillus reuteri LM1071, live bodies, heat-killed bodies, culture fluid, fragments and/or extracts thereof.

Through the whole document, the term “heat-killed bacteria” is opposite to the term “live bacteria” and refers to bodies obtained by suppressing the growth of bacteria such as heat-treating live bacteria obtained by fermentation and metabolites thereof. The heat-killed bacteria may contain cytoplasm, cell wall, antibacterial substances such as bacteriocin, polysaccharides, organic acid, and the like. Products using the heat-killed bacteria have higher stability than live bacteria products and are particularly excellent in heat resistance and have high stability to the external environment. Therefore, the products using the heat-killed bacteria are easier to store and have longer shelf life than the existing live bacteria products. Further, since the regulations on the use of antibiotics become stricter, there are a handful of companies that have produced heat-killed bacteria products. Therefore, considering the application as substitutes and the number of the producing companies, the marketability and growth potential is very high.

Through the whole document, the term “culture fluid” refers to a substance obtained by culturing the strain of the present disclosure in a known liquid medium or solid medium and may be interchangeably used with “culture medium”.

Through the whole document, the term “food” may include meats, sausages, breads, chocolates, candies, snacks, cookies, pizza, ramens, other noodles, gums, dairy products including ice cream, soups, beverages, teas, drinks, alcohol drinks, vitamin complexes, health functional foods and health foods, and may include all foods in the accepted meaning.

Through the whole document, the term “health functional food” refers to foods prepared and processed using raw materials or ingredients having useful functions to the human body in accordance with the Health Functional Food Act, No. 6727, and the “functionality” refers to adjusting nutrients on a structure and a function of the human body or obtaining a useful effect for health such as a physiological action.

The food of the present disclosure can be manufactured by conventional methods used in the art, and can be manufactured by adding conventional raw materials and ingredients used in the art. Further, a formulation of the food is not limited as long as the formulation is accepted as a food. The food composition of the present disclosure may be prepared in a variety of formulations. Since the food is used as raw materials, unlike general drugs, the food composition is free from side effects which may occur when a drug is taken for a long time, and may have excellent portability. Therefore, the food of the present disclosure may be taken as a supplement for enhancing the effects of improving the intestinal environment.

The health food refers to a food having effects of actively maintaining or promoting health conditions, as compared with general foods, and a health supplement food refers to a food for supplementing health. If necessary, the health functional food, health food and health supplement food may be interchangeably used with each other. Specifically, the health functional food is a food prepared by adding Lactobacillus reuteri LM1071 of the present disclosure to food materials such as beverages, teas, spices, gums, confectionery, etc., or prepared in a capsule, a powder or a suspension form. The health functional food means that it has a specific effect on health when consumed, but unlike general drugs, the health functional food is free from side effects that may occur when a drug is taken for a long time since the food is used as raw materials.

Since the food composition of the present disclosure can be routinely ingested, the food composition is expected to show a high efficacy on the improvement of depression and thus can be very usefully applied.

The food composition may further contain a physiologically acceptable carrier. The kind of the carrier is not particularly limited. Any carrier may be used as long as it is commonly used in the art.

Further, the food composition may further contain additional ingredients that are commonly used in food compositions so as to improve smell, taste, visuality, etc. For example, the food composition may contain vitamins A, C, D, E, B1, B2, B6, B12, niacin, biotin, folate, pantothenic acid, etc. Furthermore, the food composition may also contain minerals such as zinc (Zn), iron (Fe), calcium (Ca), chromium (Cr), magnesium (Mg), manganese (Mn), copper (Cu), chromium (Cr), etc. Moreover, the food composition may also contain amino acids such as lysine, tryptophane, cysteine, valine, etc.

Further, the food composition may also contain food additives, such as preservatives (potassium sorbate, sodium benzoate, salicylic acid, sodium dehydroacetate, etc.), disinfectants (bleaching powder, higher bleaching powder, sodium hypochlorite, etc.), antioxidants (butylhydroxyanisole (BHA), butylhydroxytoluene (BHT), etc.), colorants (tar color, etc.), color-developing agents (sodium nitrite, etc.), bleaching agents (sodium sulfite), seasonings (monosodium glutamate (MSG), etc.), sweeteners (dulcin, cyclemate, saccharin, sodium, etc.), flavors (vanillin, lactones, etc.), swelling agents (alum, potassium D-bitartrate, etc.), fortifiers, emulsifiers, thickeners (adhesive pastes), film-forming agents, gum base agents, antifoaming agents, solvents, improvers, etc. The additives may be selected and used in an appropriate amount depending on the type of food.

Lactobacillus reuteri LM1071 of the present disclosure may be added as it is, or may be used in conjunction with other foods or food ingredients, and may be appropriately used according to a conventional method. The mixing amount of active ingredients may be appropriately determined depending on the purpose of use (prophylactic, health or therapeutic treatment). In general, when a food or a beverage is manufactured, the food composition of the present disclosure may be added in an amount of 50 parts by weight or less, specifically 20 parts by weight or less based on the total weight of the food or the beverage. However, when taken for the purpose of health and hygiene, the food composition may be contained in an amount below the range. In addition, since there is no safety problem, the active ingredients may be used in an amount above the range.

The food composition of the present disclosure may be used as, for example, a health beverage composition, and in this case, the health beverage composition may further contain various flavors or natural carbohydrates, as in common beverages. The natural carbohydrates may include monosaccharides such as glucose and fructose; disaccharides such as maltose and sucrose; polysaccharides such as dextrin and cyclodextrin; and sugar alcohols such as xylitol, sorbitol and erythritol. The sweeteners may be natural sweeteners such as thaumatin or a stevia extract; or synthetic sweeteners such as saccharin or aspartame. The natural carbohydrate may be generally used in an amount of from about 0.01 g to about 0.04 g, and specifically, from about 0.02 g to about 0.03 g based on 100 mL of the health beverage composition of the present disclosure.

In addition, the health beverage composition may contain various nutrients, vitamins, minerals, flavors, colorants, pectic acid and salts thereof, alginic acid and salts thereof, organic acid, protective colloidal thickeners, pH regulators, stabilizers, antiseptics, glycerin, alcohols or carbonating agents. Moreover, the health beverage composition may contain fruit flesh used to prepare natural fruit juices, fruit juice beverages or vegetable beverages. These ingredients may be used individually or in combination. A proportion of the additives is not critical, but is generally selected from 0.01 part by weight to 0.1 part by weight per 100 parts by weight of the health beverage composition of the present disclosure.

The food composition of the present disclosure may contain Lactobacillus reuteri LM1071 of the present disclosure in a variety of % by weight as long as it can exhibit the effect of improving the intestinal environment. Specifically, Lactobacillus reuteri LM1071 of the present disclosure may be contained in an amount of 0.00001% by weight to 100% by weight or 0.01% by weight to 80% by weight based on the total weight of the food composition, but may not be limited thereto.

In an embodiment of the present disclosure, the food composition may be a health functional food composition.

A third aspect of the present disclosure provides a pharmaceutical composition for preventing or treating premenstrual syndrome, comprising one or more of a strain of Lactobacillus reuteri LM1071 (KCCM12650P), and culture fluid, fragments and extracts of the strain as active ingredients. The features described above in respect of the first and second aspects of the present disclosure may equally apply to the pharmaceutical composition according to the third aspect of the present disclosure.

In an embodiment of the present disclosure, the composition may contain a strain of Lactobacillus reuteri LM1071, live bodies, heat-killed bodies, culture fluid, culture fluid, fragments and/or extracts thereof.

Through the whole document, the term “treat” refers to all activities improving or enhancing premenstrual syndrome by administering a pharmaceutical composition containing Lactobacillus reuteri LM1071 of the present disclosure as an active ingredient to a subject with premenstrual syndrome.

In an embodiment of the present disclosure, the composition may function to treat or prevent premenstrual syndrome, and specifically, it may treat or prevent premenstrual syndrome by reducing NO, prostaglandin E2 and leukotriene in blood and increasing epoxyeicosatrienoic acid (EET) and the production ratio of prostaglandin E1/prostaglandin E2.

Through the whole document, the term “premenstrual syndrome” refers to a group of symptoms characterized by emotional, behavioral and physical symptoms that occur repeatedly before menstruation and includes edema, breast pain, digestive disorders, headache, back pain, lower abdominal pain, abdominal distention, constipation, diarrhea, depression and insomnia.

In an embodiment of the present disclosure, the pharmaceutical composition may be formulated and used as formulations for oral administration such as powders, granules, tablets, capsules, suspensions, emulsions, syrups or aerosol, external preparations, suppositories or sterile injection solutions by conventional methods, respectively, but may not be limited thereto.

In an embodiment of the present disclosure, the pharmaceutical composition may be formulated with generally used diluents or excipients such as fillers, bulking agents, binders, wetting agents, disintegrating agents or surfactants, but may not be limited thereto.

In an embodiment of the present disclosure, solid formulations for oral administration may include tablets, pills, powders, granules or capsules, and these solid formulations may be prepared by mixing heat-killed bodies of the strain with at least one of excipients such as starch, calcium carbonate, sucrose, lactose or gelatin. Except for the simple excipients, lubricants such as magnesium stearate or talc may be used, but the present disclosure may not be limited thereto.

In an embodiment of the present disclosure, liquid formulations for oral administration may include suspensions, solutions for internal use, emulsions and syrups, and may contain various excipients such as wetting agents, sweeteners, aromatics and preservatives in addition to generally used simple diluents such as water and liquid paraffin, but may not be limited thereto.

In an embodiment of the present disclosure, formulations for parenteral administration may include sterilized aqueous solutions, water-insoluble excipients, suspensions, emulsions, lyophilized preparations and suppositories, but may not be limited thereto. For example, the water insoluble excipients or suspensions may contain propylene glycol, polyethylene glycol, vegetable oil such as olive oil, injectable ester such as ethylolate, and the like, but may not be limited thereto. For example, the suppositories may contain witepsol, macrogol, tween 61, cacao butter, laurin butter, glycerol, gelatin, and the like, but may not be limited thereto.

The pharmaceutical composition according to an embodiment of the present disclosure may be a drug composition or a quasi-drug composition.

Through the whole document, the term “quasi-drug” refers to an article having a milder action than drugs, among articles being used for the purpose of diagnosis, treatment, improvement, alleviation, handling or prevention of human or animal diseases. For example, according to the Pharmaceutical Affairs Law, the quasi-drugs are those, excluding articles used as drugs, including articles used for the purpose of treating or preventing human or animal diseases and articles which have a mild action on or have no direct influence on the human body.

The quasi-drug composition of the present disclosure may be manufactured in a formulation selected from the group consisting of body cleanser, sanitizer, detergent, kitchen cleanser, detergent for cleaning, toothpaste, mouthwash, wet wipe, cleanser, soap, hand soap, hair cleanser, hair softener, humidifying filler, mask, ointment or filter filler, but may not be limited thereto.

In an embodiment of the present disclosure, the pharmaceutical composition may be administered in a pharmaceutically effective amount. Through the whole document, the term “pharmaceutically effective amount” refers to an amount sufficient to treat or prevent diseases at a reasonable benefit/risk ratio applicable to any medical treatment or prevention. An effective dosage level may be determined depending on factors including severity of the disease, drug activity, a patient's age, body weight, health conditions, gender, sensitivity to the drug, administration time, administration route, and excretion rate of the composition of the present disclosure, duration of treatment, drugs blended with or co-administered with the composition of the present disclosure, and other factors known in the medical field. The pharmaceutical composition of the present disclosure may be administered individually or in combination with an ingredient known for treating intestinal diseases. It is important to administer an amount to obtain a maximum effect in a minimum amount without side effects by considering all of the above-described factors.

In an embodiment of the present disclosure, an administration dose of the pharmaceutical composition may be determined by a person with ordinary skill in the art in view of purpose of use, severity of the disease, a patient's age, body weight, gender, medical history or the kind of a material used as an active ingredient. For example, the pharmaceutical composition of the present disclosure may be administered at a dose of from about 0.1 ng/kg to about 1,000 mg/kg, and preferably, from about 1 ng/kg to about 100 mg/kg per adult, and the administration frequency of the composition of the present disclosure is not particularly limited, but the composition may be administered once a day or several times a day in divided doses. The administration dose or the administration frequency does not limit the scope of the present disclosure in any aspect.

The pharmaceutical composition of the present application may be administered via, but not particularly limited to, intraperitoneal administration, intravenous administration, intramuscular administration, subcutaneous administration, intradermal administration, transdermal patch administration, oral administration, intranasal administration, intrapulmonary administration, rectal administration, etc. depending on the purpose. However, when the pharmaceutical composition is administered via oral administration, it can be administered in an unformulated form, and since the strain of Lactobacillus reuteri LM1071 can be denatured or degraded by gastric acid, the composition for oral administration may be coated with an active drug, formulated to be protected from degradation in the stomach, or formulated in the form or an oral patch. Also, the composition may be administered by any device capable of delivering an active ingredient to a target cell.

### MODE FOR CARRYING OUT THE INVENTION

Hereinafter, the present disclosure will be explained in more detail with reference to Examples. However, the following Examples are illustrative only for better understanding of the present disclosure but do not limit the present disclosure.

### EXAMPLES

Preparation of Composition for Relieving Premenstrual Syndrome, Containing Lactobacillus reuteri LM1071

Lactobacillus reuteri LM1071 according to the present example was prepared by batch culture.

The lactic acid bacteria were incubated in a 100 L fermenter (KoBioTech) with 60 L of culture medium, and the stirring rate was maintained at 30 rpm, the temperature was maintained at 30° C. to 37° C. and the pH was maintained at 5 to 6.5. After incubation, the culture medium was centrifuged at 13,000 rpm for 30 minutes to obtain the strain. The obtained strain was mixed with a cryoprotectant and then frozen in a cryogenic freezer for 24 hours and finally subjected to freeze-drying treatment to obtain the strain (Lactobacillus reuteri LM1071).

RAW 264.7 Cell Culture and Cytotoxicity Test

RAW 264.7 macrophage cells were incubated in RPMI 1640 containing 10% FBS. Then, for cytotoxicity test, they were seeded into a 96-well plate at a concentration of 1×10⁵ cells/well. LPS-stimulated RAW 264.7 cells were treated with Lactobacillus reuteri LM1071 at four different concentrations (10, 20, 30 and 40 μg/mL). The cell culture medium was discarded, and a cytotoxicity test was conducted using the incubated cells, and to this end, an EZ-Cytox Cell Viability Assay Kit (Daeil Labservice, Korea) was used. Cell proliferation ratio (%) was determined by the following equation.

$$Cell proliferation ratio ( \% ) = \frac{The absorbance of the test group}{The absorbance of the control group} \times 100 \qquad [ Equation ]$$

To check premenstrual pain relieving effect of a novel strain, Lactobacillus reuteri LM1071 (Depository Institution: Korean Culture Center of Microorganisms, Accession Number: KCCM12650P and Date of Deposit: Dec. 31, 2019), of the present disclosure, a test was conducted as described below.

### Example 1: Check of Change in Production Amount of Nitric Oxide (NO) by Lactic Acid Bacteria Treatment

To measure NO production ability using RAW264.7 macrophage cells, RAW264.7 cells were seeded into a 96-well plate at 1×10⁵ cells/well, and incubated in an incubator with 5% CO₂ at 37° C. for 24 hours. After 24 hours, each concentration of Lactobacillus reuteri LM1071 was added, and after incubation in the incubator with 5% CO₂ at 37° C. for 24 hours, Griess reagent was added to 100 μL of the culture supernatant and left at room temperature for 10 minutes, followed by measurement of absorbance at 540 nm with an ELISA plate reader. The concentration of nitrite, which is a metabolite of NO, was calculated from a standard curve obtained using sodium nitrite.

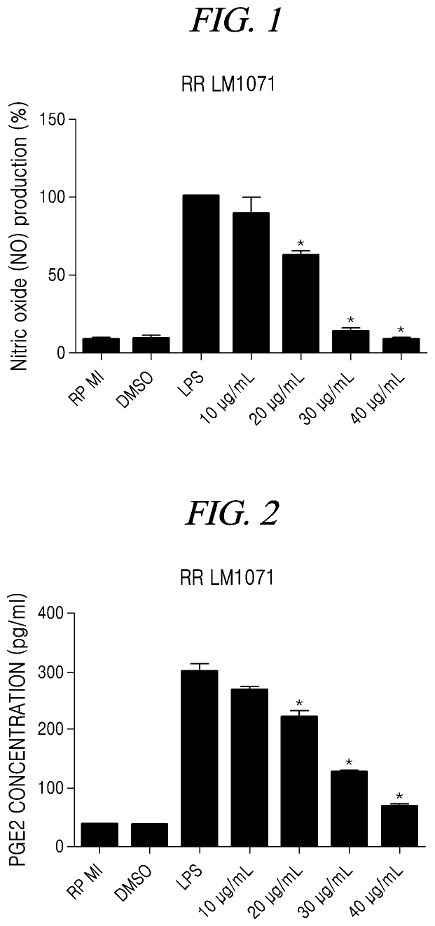

A positive control group (LPS treatment group) was set as 100% and compared with the Lactobacillus reuteri LM1071 (KCCM12650P) treatment group. As a result, a significant decrease in NO production caused by treatment with Lactobacillus reuteri LM1071 was confirmed (p<0.05). It was confirmed that when in cases of treatment with 10, 20, 30 and 40 μg/mL of Lactobacillus reuteri LM1071, NO production decreased to 87.57, 63.28, 14.26 and 8.22%, respectively, compared with the positive control group (see FIG. 1).

Based on the above result, it was confirmed that Lactobacillus reuteri LM1071 of the present disclosure reduces NO production and thus has an effect of relieving menstrual pain.

### Example 2: Check of Change in Secretion Amount of Prostaglandin by Lactic Acid Bacteria Treatment

RAW 264.7 macrophage cells were incubated in RPMI 1640 containing 10% FBS for analysis of PGE1 and PGE2 production ability and PGE1/PGE2 ratio. Also, for ELISA, they were seeded into a 96-well plate at a concentration of 1×10⁵ cells/well. LPS-stimulated RAW 264.7 cells were treated with Lactobacillus reuteri LM1071 at four different concentrations (10, 20, 30 and 40 μg/mL). The production and ratio (PGE1/PGE2) of PGE1 (prostaglandin El) and PGE2 (prostaglandin E2) were analyzed by an ELISA kit (Enzo Life Sciences, USA) using the incubated cell culture medium.

The test result confirmed that the Lactobacillus reuteri LM1071 test group showed a significant decrease in secretion amount of PGE2 from 291.16 pg/mL to 224.23, 135.13 and 71.06 pg/mL (when treated with 10, 20, 30 and 40 μg/mL of lactic acid bacteria powder, respectively) (see FIG. 2).

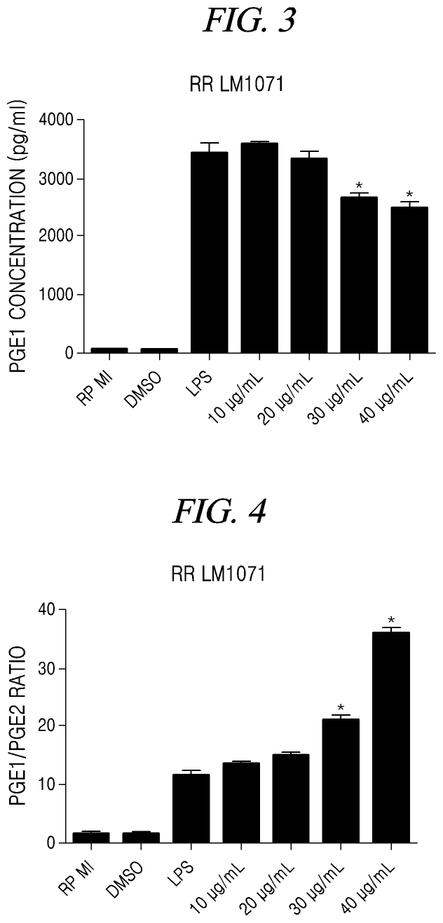

Also, the Lactobacillus reuteri LM1071 test group showed a decrease in secretion amount of PGE1 from 3467.35 pg/mL to 3062.71, 2668.48 and 2434.98 pg/mL at treatment concentrations of 10, 20, 30 and 40 μg/mL, respectively, and thus was lower in secretion amount of PGE1 by 28.37% than the positive control group (LPS treatment group) at a treatment concentration of 40 μg/mL (see FIG. 3).

Finally, a relative change in secretion of PGE2 was checked based on the ratio of PGE1 and PGE2. As a result, it was confirmed that Lactobacillus reuteri LM1071 can increase the ratio of PGE1/PGE2 to 34.31 (see FIG. 4).

Therefore, it was confirmed that Lactobacillus reuteri LM1071 is effective in relieving menstrual pain and premenstrual syndrome by reducing PGE2, which is a pain-inducing substance, and increasing the production ratio of PGE1/PGE2, which is a major PMS indicator.

### Example 3: Check of Change in Secretion Amount of Leukotriene (LTB4) and Epoxyeicosatrienoic Acid (EET) by Lactic Acid Bacteria Treatment

RAW 264.7 macrophage cells were incubated in RPMI 1640 containing 10% FBS for analysis of leukotriene (LTB4), which is a pain-inducing substance, and epoxyeicosatrienoic acid (EET), which is an inflammation-reducing substance. Also, for ELISA, they were seeded into a 96-well plate at a concentration of 1×10⁵ cells/well. LPS-stimulated RAW 264.7 cells were treated with Lactobacillus reuteri LM1071 at four different concentrations (10, 20, 30 and 40 μg/mL). The incubated cell culture medium containing LTB4 and EET was analyzed by an ELISA kit (Enzo Life Sciences, USA).

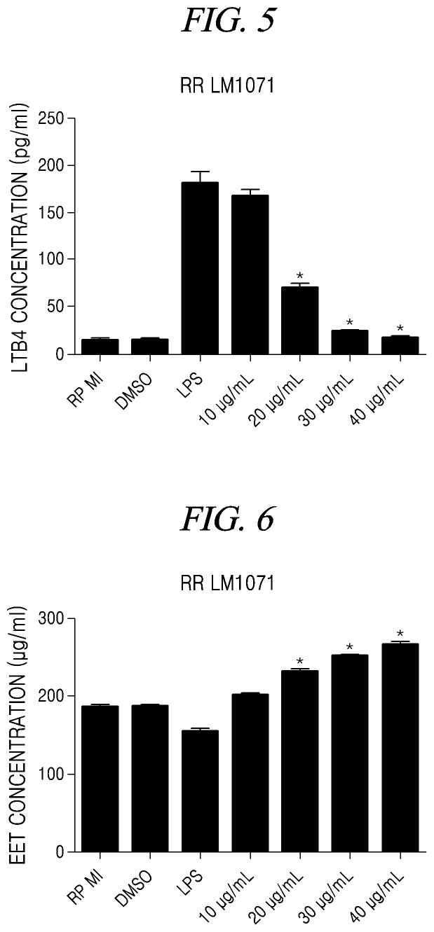

As a result of the test, the Lactobacillus reuteri LM1071 test group showed a significant decrease in LTB4 compared with the positive control group (LPS treatment group) at concentrations of 20, 30 and 40 μg/mL, and the secretion amount of LTB4 was decreased by 91.29% at a concentration of 40 μg/mL and measured at 15.75 pg/mL (see FIG. 5).

Alto, the Lactobacillus reuteri LM1071 test group showed a higher concentration of EET secretion than the positive control group (LPS treatment group), which confirmed the possibility of reliving pain through inhibition of inflammatory responses. Lactobacillus reuteri LM1071 showed an increase in secretion amount of EET to 184.79, 190.25, 218.98 and 268.41 μg/mL depending on treatment concentration (see FIG. 6).

Accordingly, it was confirmed that Lactobacillus reuteri LM1071 suppresses production of leukotriene and increases the secretion amount of epoxyeicosatrienoic acid (EET) and thus shows efficacy in relieving inflammation and pain.

Overall, it was confirmed that Lactobacillus reuteri LM1071 can contribute to relieving menstrual pain by inhibiting NO and PGE2 and LTB4, which are pain-inducing substances, increasing the PGE1/PGE2 ratio, and promoting production of EET, which is an inflammation-reducing substance.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure. Accession Number

### ACCESSION NUMBER

Depository Institution: Korean Culture Center of Microorganisms (Foreign Country)

Accession Number: KCCM12650P

Date of Deposit: Dec. 31, 2019

We claim:

1. A strain of Lactobacillus reuteri LM1071 (KCCM12650P).

2. The strain of claim 1, wherein the strain reduces the production amount of NO in blood.

3. The strain of claim 1, wherein the strain reduces the secretion amount of prostaglandin E2 in blood.

4. The strain of claim 1, wherein the strain increases the ratio of prostaglandin E1/prostaglandinE2 in blood.

5. The strain of claim 1, wherein the strain reduces production of leukotriene in blood.

6. The strain of claim 1, wherein the strain increases production of epoxyeicosatrienoic acid (EET) in blood.

7. A food composition for relieving menstrual pain, comprising:
one or more of a strain of Lactobacillus reuteri LM1071 (KCCM12650P), and culture fluid, fragments and extracts of the strain as active ingredients.

8. A pharmaceutical composition for preventing or treating premenstrual syndrome, comprising:
one or more of a strain of Lactobacillus reuteri LM1071 (KCCM12650P), and culture fluid, fragments and extracts of the strain as active ingredients.

9. The pharmaceutical composition for preventing or treating premenstrual syndrome of claim 8,
wherein the premenstrual syndrome includes edema, breast pain, digestive disorders, headache, back pain, lower abdominal pain, abdominal distention, constipation, diarrhea, depression and insomnia.
